(19) United States
(12) Patent Application Publication
(10) Pub. No.: US 2013/0060099 A1
(43) Pub. Date: Mar. 7, 2013

(54) Methods for Subcutaneously Positioning an Analyte Sensing Device

(71) Applicants: Adam Heller, Austin, TX (US); Keith A. Friedman, Austin, TX (US)

(72) Inventors: Adam Heller, Austin, TX (US); Keith A. Friedman, Austin, TX (US)

(73) Assignee: Abbott Diabetes Care Inc.

(21) Appl. No.: 13/599,437

(22) Filed: Aug. 30, 2012

Related U.S. Application Data
(60) Provisional application No. 61/529,138, filed on Aug. 30, 2011.

(51) Int. Cl. A61B 5/1473; A61B 5/11; A61B 5/145

(52) U.S. Cl. 600/301; 600/309; 600/345; 600/347

(57) ABSTRACT

Aspects of the present disclosure include methods for determining the presence and/or concentration of an analyte. In practicing methods according to certain embodiments, an analyte sensing unit is positioned at a location on the abdomen of a that experiences involuntary movement sufficient to provide for mixing of non-circulating interstitial fluid with circulating interstitial fluid and determining an analyte concentration in the interstitial fluid. Also provided are methods for positioning an analyte sensing unit at a location on the abdomen of a subject, and methods of determining an analyte concentration while the subject is asleep, e.g., during a rapid eye movement (REM) sleep period. Devices and systems for practicing the subject methods also described.

### CROSS-REFERENCE TO RELATED APPLICATION

This application claims the benefit of priority under 35 U.S.C. §119(e) to U.S. Provisional Patent Application No. 61/529,138 filed Aug. 30, 2011, the disclosure of which is incorporated by reference herein in its entirety.

### INTRODUCTION

Management of diabetes requires knowledge of the glycemia of patients. In general, health care professionals and diabetic patients base their decisions of injection and dosage of insulin or ingestion of food on blood glycemia, meaning the glucose concentration in blood. In hospitals or clinics, venous blood is withdrawn and sent to a laboratory for analysis or is analyzed at the bedside or in the office of the health care professional. Many times, however, the skin is lanced by the diabetic patient to obtain a droplet of blood which is used for a glucose assay such as with a glucose test strip system. Systems for frequently or continuously and automatically monitoring glycemia in the subcutaneous ISF, known as continuous glucose monitoring (CGM) devices, are also available.

While continuous glucose monitoring is desirable, there are several challenges associated with obtaining accurate and stable glucose concentrations from continuous glucose monitors in interstitial fluid. Accordingly, further development of methods for obtaining accurate glucose concentrations from interstitial fluid as well as analyte-monitoring devices and systems is desirable.

### SUMMARY

Aspects of the present disclosure include methods for determining an analyte concentration. In practicing methods according to certain embodiments, an analyte sensing unit is positioned at a location on the abdomen of a subject, such that the location experiences involuntary movement sufficient to provide for mixing of non-circulating interstitial fluid with circulating interstitial fluid and determining an analyte concentration in the interstitial fluid. Also provided are methods for positioning an analyte sensing unit at a subcutaneous location on the abdomen of a subject and methods of determining an analyte concentration while the subject is asleep. Devices and systems for practicing the subject methods are also described.

### BRIEF DESCRIPTION OF THE DRAWINGS

A detailed description of various embodiments of the present disclosure is provided herein with reference to the accompanying drawings, which are briefly described below. The drawings are illustrative and are not necessarily drawn to scale. The drawings illustrate various embodiments of the present disclosure and may illustrate one or more embodiment(s) or example(s) of the present disclosure in whole or in part. A reference numeral, letter, and/or symbol that is used in one drawing to refer to a particular element may be used in another drawing to refer to a like element.

FIG. 1 shows a schematic of suitable positions on the abdomen of a human according to certain embodiments of the present disclosure.

FIGS. 2A-2E show histograms from sensors positioned according to certain embodiments of the present disclosure.

FIGS. 3A-3E show histograms from sensors positioned according to certain embodiments of the present disclosure.

FIGS. 4A-4D show correlation data and histograms from sensors positioned according to certain embodiments of the present disclosure.

FIGS. 5A-5B show correlation data between continuous glucose monitoring sensors in the interstitial fluid and blood glucose values.

FIGS. 6A-6F show correlation data between continuous glucose monitoring sensors in the interstitial fluid and blood glucose values positioned at varying locations on the abdomen according to certain embodiments.

FIGS. 7A-7B show correlation data between continuous glucose monitoring sensors in the interstitial fluid and blood glucose values positioned at varying locations on the abdomen according to certain embodiments.

### DETAILED DESCRIPTION

Aspects of the present disclosure include methods for determining the presence and/or concentration of an analyte. In practicing methods according to certain embodiments, an analyte sensing unit is positioned at a location on the abdomen of a that experiences involuntary movement sufficient to provide for mixing of non-circulating interstitial fluid with circulating interstitial fluid and determining an analyte concentration in the interstitial fluid. Also provided are methods for positioning an analyte sensing unit at a location on the abdomen of a subject, and methods of determining an analyte concentration while the subject is asleep, e.g., during a rapid eye movement (REM) sleep period. Devices and systems for practicing the subject methods also described.

Before the embodiments of the present disclosure are described, it is to be understood that this invention is not limited to particular embodiments described, as such may, of course, vary. It is also to be understood that the terminology used herein is for the purpose of describing particular embodiments only, and is not intended to be limiting, since the scope of the embodiments of the invention will be embodied by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

In the description of the invention herein, it will be understood that a word appearing in the singular encompasses its plural counterpart, and a word appearing in the plural encompasses its singular counterpart, unless implicitly or explicitly understood or stated otherwise. Merely by way of example, reference to “an” or “the” “analyte” encompasses a single analyte, as well as a combination and/or mixture of two or more different analytes, reference to “a” or “the” “concentration value” encompasses a single concentration value, as well as two or more concentration values, and the like, unless implicitly or explicitly understood or stated otherwise. Further, it will be understood that for any given component described herein, any of the possible candidates or alternatives listed for that component, may generally be used individually or in combination with one another, unless implicitly or explicitly understood or stated otherwise. Additionally, it will be understood that any list of such candidates or alternatives is merely illustrative, not limiting, unless implicitly or explicitly understood or stated otherwise.

Various terms are described below to facilitate an understanding of the invention. It will be understood that a corresponding description of these various terms applies to corresponding linguistic or grammatical variations or forms of these various terms. It will also be understood that the invention is not limited to the terminology used herein, or the descriptions thereof, for the description of particular embodiments. Merely by way of example, the invention is not limited to particular analytes, bodily or tissue fluids, blood or capillary blood, or sensor constructs or usages, unless implicitly or explicitly understood or stated otherwise, as such may vary.

The publications discussed herein are provided solely for their disclosure prior to the filing date of the application. Nothing herein is to be construed as an admission that the embodiments of the invention are not entitled to antedate such publication by virtue of prior invention. Further, the dates of publication provided may be different from the actual publication dates which may need to be independently confirmed.

In further describing the present disclosure, methods for determining an analyte concentration in a subject are described first in greater detail. Next, devices and systems practicing methods of the present disclosure are also described.

### Methods for Monitoring an Analyte Using a Sensor Unit Positioned on the Abdomen of a Subject

As summarized above, aspects of the disclosure include methods for determining the presence and/or concentration of an analyte in a subject by subcutaneously positioning at least a portion of an analyte sensing device at a location on the abdomen of the subject, where the location predetermined for a positioned sensing device experiences localized involuntary movement sufficient to provide for mixing of non-circulating interstitial fluid with circulating interstitial fluid, such that mixing of the non-circulating interstitial fluid with circulating interstitial fluid is greater than at other areas of the abdomen that do not experience localized involuntary movement, and determining an analyte concentration in the interstitial fluid. Embodiments include determining abdominal positions that provide for greater or optimal mixing of non-circulating interstitial fluid with circulating interstitial fluid, such that the mixing of the non-circulating interstitial fluid with circulating interstitial fluid is greater at the optimal mixing areas than at other abdominal areas of the abdomen.

Interstitial fluid circulation is the movement of fluid through a three dimensional extracellular matrix of tissue and is present in all tissues where convection is needed to transport solutes through the interstitial space. Incoming interstitial fluid originates in the arterioles and is rapidly cleared primarily by the venules. However, in some cases, interstitial fluid which is not cleared by venules is cleared more slowly by the lymphatic system. As a result, interstitial fluid not rapidly cleared by the venules often remains stagnant (i.e., is non-circulating or experiences little to no movement) in certain parts of the body resulting in spatially and temporally heterogenous concentration of the analyte between the non-circulating interstitial fluid and the circulating interstitial fluid. In other words, the non-circulating interstitial fluid may have a different analyte concentration than the circulating interstitial fluid or the analyte concentration in the non-circulating interstitial fluid may lag behind the analyte concentration in the circulating interstitial fluid. In addition, the analyte concentration in the non-circulating interstitial fluid may not strongly correlate with the analyte concentration in blood. However, when the non-circulating interstitial fluid is mixed with the circulating interstitial fluid by localized involuntary muscle movement, the analyte concentration in the non-circulating interstitial fluid will become homogeneous with the analyte concentration in the circulating interstitial fluid and will strongly correlate with the analyte concentration in the blood.

In embodiments of the present disclosure, positive identification of an abdominal area is provided at which movement by the abdomen is sufficient to provide for mixing of non-circulating interstitial fluid with circulating interstitial fluid in the abdominal interstitial space. An analyte sensor device may be subcutaneously positioned at a location on the abdomen such that involuntary muscle movements sufficiently mix non-circulating interstitial fluid in close proximity to the sensor with the circulating interstitial fluid. This involuntary movement ensures that the interstitial fluid in contact with the subcutaneously positioned analyte sensor that is not rapidly cleared by the venules (i.e., non-circulating interstitial fluid) does not remain stagnant, i.e., remains in motion or is dynamic. As such, the interstitial fluid remains stagnant in the interstitial space for 10 minutes or less, such as 8 minutes or less, such as 6 minutes or less, such as 5 minutes or less, such as 4 minutes or less, such as 3 minutes or less, such as 2 minutes or less including 1 minute or less. In some embodiments, a sufficient mixing of the non-circulating interstitial fluid with the circulating interstitial fluid may be provided by a correspondingly sufficient amount of movement by the abdomen.

The glycemia of interstitial fluid at a location where interstitial fluid is circulating (i.e., rapidly cleared by the venules) or where non-circulating interstitial fluid is mixed with the circulating interstitial fluid by movement at the location correlates strongly with instantaneous blood glycemia. On the other hand, the glycemia of non-circulating interstitial fluid at a location where the interstitial fluid is not rapidly cleared by the venules or is not mixed with the circulating interstitial fluid by movement at the location (i.e., is stagnant) can result in a poor correlation between glycemia in interstitial fluid and instantaneous blood glycemia.

In certain embodiments, the method further includes monitoring or measuring (automatically) the localized involuntary muscle movement sufficient to provide for mixing of circulating and non-circulating interstitial fluid in a subcutaneous space of the location, during the sensor wear period. In such embodiments, a motion sensor may be positioned proximal to the analyte sensor and may measure or monitor the level of localized involuntary muscle movement to determine whether the level of movement meets a predetermined level that provides for mixing of circulating and non-circulating interstitial fluid in a subcutaneous space of the location, during the sensor wear period.

Aspects of the present disclosure include methods for determining an analyte concentration in the interstitial fluid of a subject such that the analyte concentration in the interstitial fluid correlates strongly with the concentration of the analyte in the blood. In some instances, methods include subcutaneously positioning an analyte sensing device at a location on the abdomen where the location experiences localized involuntary muscle movement sufficient to provide for mixing of the interstitial fluid such that analyte concentration in the interstitial fluid strongly correlates with the analyte concentration in the blood.

In embodiments of the present disclosure, mixing of interstitial fluid at the location where the analyte sensor unit is positioned may be sufficient when the concentration of the analyte in the interstitial fluid at the location on the body correlates strongly with the concentration of the analyte as determined in the blood using a standard blood glucose test such as for example, by glucose test strip. By “correlates strongly” is meant that at least 80% or more of the concentration values determined from the interstitial fluid are within 20% or more of the concentration values as determined in blood as described in Clarke et al., Diabetes Care, Vol. 10(5):622-628 (1987). For example, at least 85% or more, such as 88% or more, such as 90% or more, such as 95% or more, such as 98% or more, and including 99% or more of the concentration values determined from the interstitial fluid are within 20% or more, such as within 15% or more, such as within 10% or more, including within 5% or more of the concentration values as determined by blood. In certain embodiments, at least 95% of the concentration values of the analyte determined in the interstitial fluid are within 5% of the concentration values as determined in blood.

Involuntary Muscle Movements of the Abdomen

In some embodiments, methods for determining an analyte concentration in a subject include predetermining a location on the abdomen where the location experiences localized involuntary muscle movement sufficient to provide for the mixing of non-circulating interstitial fluid with circulating interstitial fluid and determining an analyte concentration in the mixed interstitial fluid, positioning an analyte sensing device at the predetermined location on the abdomen, and determining an analyte concentration in the interstitial fluid. As described herein, the term “involuntary muscle movement” is used to refer to muscle movement which occurs without conscious thought or intention. As such, involuntary muscle movements are movements which occur without intentional control by the subject and are in some instances, movements which are essential to maintaining bodily homeostasis or are necessary for survival. Examples of involuntary muscle movement may include, but are not limited to contractions by the heart, peristalsis of the digestive system and contraction of the diaphragm during breathing, among others. In some embodiments, involuntary muscle movement is associated with or is the result of the contraction of involuntary muscle groups in the subject. For example, involuntary muscles (i.e., “smooth muscle”) may be found within the walls of internal organs and bodily structures such as the esophagus, stomach, intestines, bronchi, uterus, urethra, bladder, blood vessels, among other. In embodiments of the present disclosure, involuntary muscle movements may be movements which occur according to physiologically predetermined time intervals. For example, in some instances, the involuntary muscle movement occurs every 1 second or more, such as every 2 seconds or more, such as every 5 seconds or more, such as every 10 seconds or more, such as every 15 seconds or more, including every 30 seconds or more.

In certain embodiments, the involuntary muscle movement may be movement that is associated with respiration or normal breathing. The term “breathing” is used herein in its conventional sense to refer to the process of moving air into (i.e., inhaling) the lungs by the contraction of the diaphragm muscle to increase thoracic volume and moving air out (i.e., exhaling) of the lungs by relaxation of the diaphragm muscle to decrease thoracic volume. Normal breathing is an unconscious movement controlled by the brainstem, which automatically regulates the rate and depth of breathing depending upon the body's needs. Localized movement of the body during breathing may vary depending on the physiology of the subject as well as the rate and depth of breathing. By involuntary muscle movement associated with breathing is meant movement of the body during normal unconscious breathing and is distinct from intentional (i.e., conscious) manipulations of breathing such as taking a deep breath or intentional hyperventilation which employ secondary muscle groups to consciously change the pattern of breathing.

Aspects of the present disclosure include subcutaneously positioning an analyte sensor device at a location on the abdomen of a subject such that the location experiences localized involuntary movement sufficient to provide for mixing of non-circulating interstitial fluid with circulating interstitial fluid at the location on the abdomen. The term “localized” is used in its conventional sense to refer to movement which is within 50 mm or less from the location of the positioned sensor. For example, movement as provided by embodiments of the disclosure may include movement which is 45 mm or less, such as 40 mm or less, such as 35 mm or less, such as 30 mm or less, such as 25 mm or less, such as 20 mm or less, such as 15 mm or less, such as 10 mm or less, including 5 mm or less from the location of the analyte sensor device.

As analyte sensing devices of the present disclosure are subcutaneously positioned at a location on the abdomen which experiences localized involuntary muscle movement, the involuntary muscle movements of the abdomen are, in certain embodiments, sufficient to enable the mixing of the non-circulating interstitial fluid with the circulating interstitial fluid and as a result enable the determination of an analyte concentration in the interstitial fluid at the location on the abdomen which correlates closely with the analyte concentration as determined in the blood. However, the analyte sensing device is not affected by the localized involuntary muscle movements of the abdomen other than being in contact with interstitial fluid that has an analyte concentration that more closely correlates with analyte concentration in the blood.

In embodiments, the analyte sensor device is positioned on the involuntarily moving part of the abdomen of the subject. As used herein, the term abdomen (i.e., belly) refers to the part of the body located between the thorax and pelvis. The abdomen may be divided into regions, including the central abdomen and the outer abdomen. The outer abdomen may include the lower abdomen situated near the pelvis and the upper abdomen situated near the lower thorax. The outer abdomen also includes the part of the abdomen distal to the midline of the body on either the right or left side of the body.

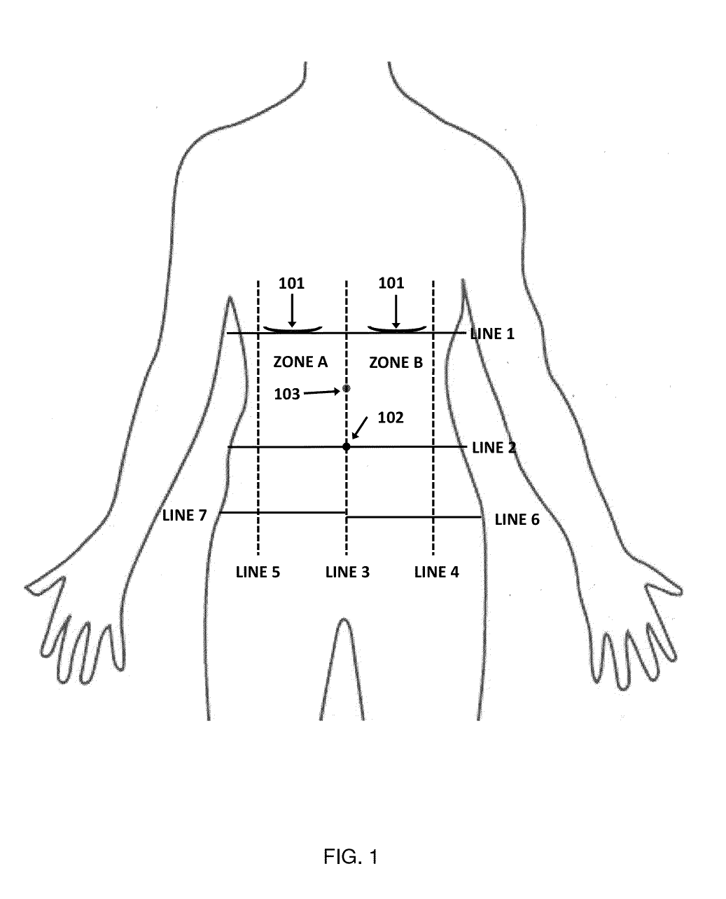

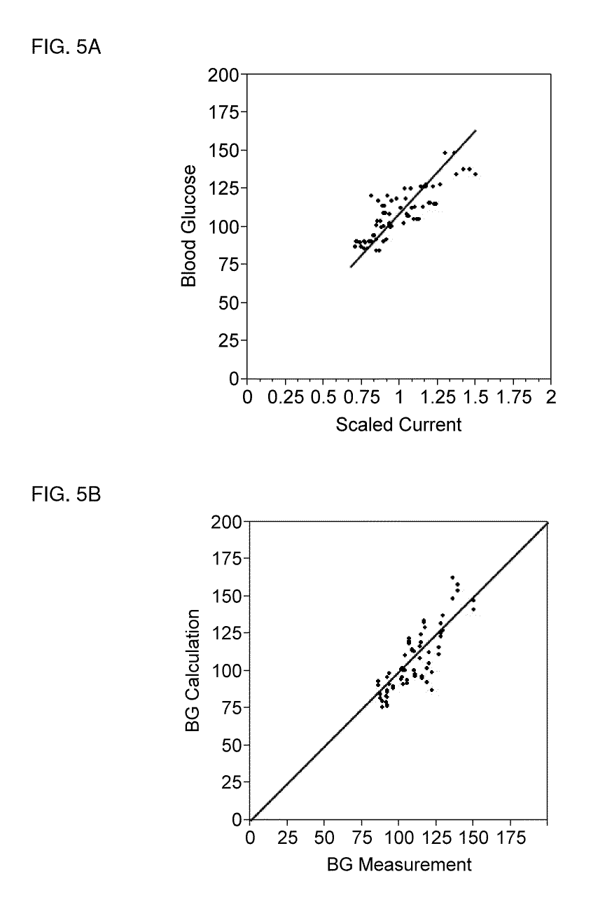

FIG. 1 depicts certain locations for subcutaneously positioning an analyte sensor device on the abdomen according to methods of the present disclosure. In some instances, locations on the abdomen suitable for placing an analyte sensing device include two zones (e.g., Zones A and B in FIG. 1) defined by a first line (LINE 1) connecting the two lowest points of the ribcage (101); a second line (LINE 2) parallel to the first line which extends through the navel (102) and three lines orthogonal to the first and second line (LINES 3-5). LINE 3 of FIG. 5 extends along the midline of the body through the navel and connects LINES 1 and 2. As such, in some embodiments, LINE 1, connecting the two lowest points on the ribcage forms the upper boundary and LINE 2, extending through the navel forms the lower boundary. Likewise, LINE 4 forms a first lateral boundary and LINE 5 forms a second lateral boundary. In these embodiments, the apex of the abdomen (i.e., the central point of the abdomen, 603) is the point equidistant from the upper boundary (i.e., LINE 1) and the lower boundary (i.e., LINE 2) along the midline of the body (i.e., LINE 3).

The physiology of subjects employing the methods described herein may vary depending on many factors such as age, gender, height and weight. As such, locations for positioning an analyte sensor device on the abdomen according to embodiments of the disclosure may vary. As described above, locations for positioning an analyte sensor device on the abdomen may include locations which are located below the lowest points of the ribcage and above the level of navel (e.g., Zones A and/or B as depicted in FIG. 1). Depending on the physiology of the subject, suitable locations on the abdomen which are located below the lowest points of the ribcage and above the level of navel and may extend laterally (i.e., from LINE 3 to LINES 4 and 5 as illustrated in FIG. 1) across the body up to about 75% of the distance from the midline of the body to the hip joint (i.e., 75% of LINES 6 or 7), such as up to 65% of the distance from the midline of the body to the hip joint, such as up to 50% of the distance from the midline of the body to the hip joint, such as up to 35% of the distance from the midline of the body to the hip joint, including up to 25% of the distance from the midline of the body to the hip joint. For example, in certain instances, suitable locations (i.e., Zones A and/or B) may extend laterally across the body up to about 12 cm or less from the midline of the body, such as 11 cm or less, such as 10 cm or less, such as 8 cm or less, such as 5 cm or less, including 3 cm or less from the midline of the body.

In certain embodiments, the analyte sensor device is positioned relative to the apex of the abdomen. As described above, the apex of the abdomen is the central point of the abdomen, situated along the midline of the body (i.e., LINE 3 in FIG. 1 which separate Zones A and B) and equidistant from the lowest point of the ribcage (i.e., LINE 1) and the navel (i.e., LINE 2). In some embodiments, an analyte sensor device is positioned within about 12 cm or less from the apex of the abdomen, such as within about 11 cm or less, such as within about 10 cm or less, such as within about 9 cm or less, such as within about 8 cm or less, such as within about 7 cm or less, such as within 6 cm or less, such as within about 5 cm or less, such as within about 4 cm or less, including within about 3 cm or less of the apex of the abdomen.

The analyte sensor device may also be positioned relative to the navel. Where the analyte sensor device is positioned near the navel, the analyte sensor device may be positioned above the navel as desired, depending on the movement and physiology of the subject. As such, the analyte sensor device may be positioned within about 12 cm or less above the navel, such as within about 11 cm or less, such as within about 10 cm or less, such as within about 9 cm or less, such as within about 8 cm or less, such as within about 7 cm or less, such as within 6 cm or less, such as within about 5 cm or less, such as within about 4 cm or less, including within about 3 cm or less above the navel.

The analyte sensor device may also be positioned relative to the diaphragm of the subject. The term diaphragm is used in its conventional sense to refer to the internal muscle extending across the bottom of the rib cage, which separates the thorax from the abdomen. As such, the diaphragm is the border between the abdomen and the thorax. As noted above, the diaphragm may be the upper border (i.e., LINE 1 which connects the bottom points of the ribcage) of Zones A and B illustrated in FIG. 1. Where the analyte sensor device is positioned in relation to the diaphragm depending on the physiology of the subject, the analyte sensor device may be positioned within about 12 cm or less below the diaphragm, such as about 11 cm or less below the diaphragm, such as about 10 cm or less below the diaphragm, such as about 9 cm or less below the diaphragm, such as about 8 cm or less below the diaphragm, such as about 7 cm or less below the diaphragm, such as about 6 cm or less below the diaphragm, such as about 5 cm or less below the diaphragm, such as about 4 cm or less below the diaphragm, including about 3 cm or less below the diaphragm.

In embodiments of the present disclosure, localized involuntary movement at a location on the abdomen of a subject may include movement that is the result of spontaneous inhalation and exhalation. In other words, localized involuntary movement may be the displacement (i.e., rise and fall) of the abdomen during breathing. Localized movement may be described in terms of its “amplitude of displacement” or “total displacement” which is the sum total of distance traversed by the abdomen during movement. For example, by the abdomen having a total displacement of 2 mm is meant the abdomen traverses a total of 2 mm during the particular localized movement. In some instances, the abdomen may move 2 mm from its initial location and come to a stop or in other instances, the abdomen may move 1 mm from its initial location and move a second 1 mm to return to its initial location for a total of 2 mm traversed.

Depending on the depth of breathing by the subject, the amplitude of displacement of the abdomen during breathing may range, such as from about 10 to 75 mm, such as from about 15 to 65 mm, such as from about 20 to 60 mm, such as from about 25 to 55 mm, such as from about 25 to 50 mm, including from about 25 to 45 mm. Movement of the abdomen during breathing also varies depending on the respiratory rate the subject. The respiratory rate may range, such as for example from about 8 to 22 breaths (i.e., cycles of inhalation and exhalation) per minute, such as about 10 to 20 breaths per minute, such as about 12 to 18 breaths per minute, such as about 12 to 15 breaths per minute, including about 14 breaths per minute. As such, the total localized movement as a result of the displacement of the abdomen during breathing may be from about 50 to about 1000 mm per minute, such as from about 75 to 750 mm per minute, such as from about 100 to 500 mm per minute, such as from about 150 to 400 mm per minute, including about 250 mm per minute.

In embodiments of the present disclosure, prior to positioning the analyte sensor device on the abdomen, a specific location on the abdomen is identified and selected as a suitable location for positioning the analyte sensor device. Any convenient location on the abdomen may be suitable for positioning the analyte sensor device according to the present disclosure so long as the selected location or locations experiences localized involuntary movement sufficient to provide for equilibration of stagnant interstitial at the location. In certain instances, a location on the abdomen is suitable for positioning the analyte sensor device because the location experiences localized involuntary movement sufficient to provide for the steady mixing of interstitial fluid at the location. In identifying and selecting a suitable location on the abdomen for positioning the analyte sensor device, methods may further include determining the amplitude (e.g., rate of displacement) of involuntary muscle movement at the desired location on the abdomen. For example, the amplitude of movement of the abdomen during breathing may be determined, as discussed above. In other instances, identifying and selecting a suitable location on the abdomen for positioning the analyte sensor unit may include determining the flow rate of interstitial circulation at the desired location.

In certain embodiments, selecting a location includes one or more of locating the navel of the subject, locating the lowest point of the ribcage of the subject, locating the midline of the body of the subject, and locating a position along the midline which is equidistant from the lowest point of the ribcage and the navel of the subject. In certain instances, selecting a location for positioning the analyte sensor device includes locating the lowest point of the ribcage of the subject. As described above, the lowest point of the ribcage may be defined by a first line connecting the two lowest points of the ribcage (LINE 1 of FIG. 1) which extends laterally across the body and through the midline of the body. In other instances, selecting a location for positioning the analyte sensor device also includes locating the navel of the subject.

Depending on the physiology of the subject, selecting a location for positioning an analyte sensor device may include locating a position that is equidistant superiorly from the navel and inferiorly from the lowest point of the ribcage along the midline of the abdomen (i.e., apex of the abdomen). In other instances, selecting a location for positioning the analyte sensor device may include locating a position equidistant superiorly from the navel and inferiorly from the lowest point of the ribcage that is 75% or less of the lateral distance to the left or right from the midline of the body to the hip joint, such as 65% or less, such as 50% or less, such as 35% or less, and including 25% or less of the lateral distance to the left or right from the midline of the body to the hip joint. In other instances, selecting a location for positioning the analyte sensor device may include locating a position equidistant superiorly from the navel and inferiorly from the lowest point of the ribcage and is laterally displaced 12 cm or less to the left or to the right from the midline of the abdomen of the subject, such as 11 cm or less, such as 10 cm or less, such as 9 cm or less, such as 7 cm or less, such as 5 cm or less, such as 3 cm or less and including 2 cm or less to the left or to the right from the midline of the abdomen of the subject. In certain instances, selecting a location for positioning an analyte sensor device may include selecting a location which is defined by Zone A and/or Zone B according to FIG. 5 as described in detail above. In these instances, selecting a location for positioning the analyte sensor device may include selecting a location that is in Zone A and/or Zone B according to FIG. 5 such that the location is superior to the navel, inferior to the lowest point of the ribcage and is 75% or less of the lateral distance to the left or right from the midline of the body to the hip joint, such as 65% or less, such as 50% or less, such as 35% or less, and including 25% or less of the lateral distance to the left or right from the midline of the body to the hip joint. In other instances, selecting a location for positioning the analyte sensor device may include selecting a location that is in Zone A and/or Zone B according to FIG. 5 such that the location is superior to the navel, inferior to the lowest point of the ribcage and is laterally displaced 12 cm or less from the midline of the body, such as 11 cm or less, such as 10 cm or less, such as 8 cm or less, such as 5 cm or less, including 3 cm or less from the midline of the body.

Nighttime Dropout

Another aspect of the present disclosure includes reliably determining an analyte concentration using an analyte sensor unit while the subject is asleep by positioning an analyte sensor device at a location on the abdomen of the subject such that the abdomen experiences localized involuntary movement during sleep, sufficient to provide for mixing of non-circulating interstitial fluid with circulating interstitial fluid at the location and determining an analyte concentration in the interstitial fluid. The term “asleep” is used in its conventional sense to refer to a state characterized by reduced or absent consciousness, relative suspended sensory activity and inactivity of voluntary muscle movements. As such, the term “asleep” as used herein may also include naturally-occurring states such as being in hibernation or in a coma. The term “asleep” may also refer to induced states of reduced or absent consciousness and inactivity of voluntary muscle movements, such as for example, placing a subject under general anesthesia. As noted above, during sleep, activity of voluntary muscle movements is reduced or entirely suspended. As such, movement of the body during sleep is largely due to involuntary muscle movements such as movements associated with respiration. Therefore, methods of the present disclosure may also include determining an analyte concentration while the subject is asleep by positioning an analyte sensor unit at a location on the abdomen of the subject such that the location experiences localized involuntary muscle movement during sleep, sufficient to provide for mixing of interstitial fluid at the location. Therefore, since the location on the abdomen for subcutaneously positioning the analyte sensor device experiences localized involuntary muscle movement during sleep, these sensors produce superior accuracy as compared to other sensors which are positioned at locations on the body which experience little or no movement during sleep. By superior accuracy is meant that analyte sensor devices positioned according to methods of the present disclosure generate analyte concentration values which correlate with analyte concentration values as determined by blood 50% better or more than sensors which are positioned at locations on the body which experience little or no movement during sleep, such as 60% better or more, such as 75% better or more, such as 90% better or more, such as 95% better or more, including 99% better or more than sensors which are positioned at locations on the body which experience little or no movement during sleep. Since other sensors are positioned at locations which experience little or no movement during sleep, the interstitial fluid remains stagnant and produces spatially and temporally heterogenous concentration measurements of analytes.

As such, methods of the present disclosure help to reduce hypoglycemic events and false hypoglycemia alarms during sleep or periods of little to no deliberate or voluntary muscle movement. Furthermore, methods of the present disclosure may in some instances help to prevent “nighttime dropoff” of glucose values by continuous glucose monitoring devices which may simply be the result of reduced mixing of non-circulating and circulating interstitial fluid and not necessarily a decrease in actual blood glucose. Since the analyte sensor device is positioned at a location on the abdomen which experiences consistent (and continuous) localized involuntary muscle movement, methods of the present disclosure remedy problems associated with reduced interstitial fluid mixing which may produce the inaccurate dropoff of glucose concentration values measured during sleep.

In embodiments of the present disclosure, locations on the abdomen for positioning an analyte sensor device during sleep may vary, and may include locations on the abdomen such as those described in detail above. In some instances the analyte sensor device is positioned at the same location during sleep and during awake hours. In other embodiments, the analyte sensor device may be positioned at different locations of the abdomen during sleep and during awake hours. For example, during awake hours, the analyte sensor device may be positioned at 12 cm or less from the apex of the abdomen, such as 10 cm or less from the apex of the abdomen, such as 9 cm or less from the apex of the abdomen including 8 cm or less from the apex of the abdomen. On the other hand, during nighttime sleep hours, the analyte sensor device may be positioned at 5 cm or less from the apex of the abdomen, such as 4 cm or less from the apex of the abdomen, including 3 cm or less from the apex of the abdomen.

In embodiments of the present disclosure, an analyte sensor unit is positioned at a location on the abdomen of a subject. In some instances, positioning an analyte sensor unit at a location on the abdomen includes positioning at least a portion of an analyte sensor in the subcutaneous tissue at the abdomen. As described below, force may be applied to an insertion device, either manually or mechanically, to position at least a portion of the sensor beneath the surface of the skin. In certain instances, an insertion device may be employed to implant the sensor into the subcutaneous tissue. Insertion devices for implanting an analyte sensor into the subcutaneous tissue may include, but are not limited to those described in U.S. Pat. No. 6,175,752 filed Apr. 30, 1998, the disclosure of which is incorporated by reference in its entirety. The depth of implantation varies depending on the physiology of the subject as well as the particular location on the body selected. As such, the analyte sensor may be implanted to a depth of from about 1.0 to 15.0 mm beneath the surface of the skin, such as about 1.5 to 12.5 mm, such as about 2.0 to 10.0 mm, such as about 2.5 to 9.0 mm, such as 3.0 to 7.5 mm, including 4.0 to 6.0 mm beneath the surface of the skin.

In certain embodiments, methods of the present disclosure include positioning more than one analyte sensor device on the abdomen of the subject. Where more than one analyte sensor devices are positioned on the abdomen, the analyte sensor devices may be positioned on the same side of the abdomen with respect to the midline of the body (e.g., all in Zone A of FIG. 5) or on opposite sides of the abdomen with respect to the midline of the body (e.g., one in Zone A and one is Zone B of FIG. 5) or any combination thereof. For example, in certain instances, two or more analyte sensor device may be positioned on the abdomen, such as for example, a first analyte sensor device on the left side of the abdomen and a second analyte sensor device on the right side of the abdomen. In other instances, a first analyte sensor device is positioned within 5 cm below the apex of the abdomen along the midline of the body and a second analyte sensor device is positioned within 5 cm above the apex of the abdomen along the midline of the body. In yet other instances, two or more analyte sensor devices may be positioned on the abdomen, such as for example a first analyte sensor device positioned within 12 cm to the left of the apex of the abdomen and a second analyte sensor device positioned within 12 cm to the right side of the apex of the abdomen or any combination thereof.

In certain instances, methods of the present disclosure further include determining the concentration of an analyte using two or more analyte sensor devices positioned at different locations of the abdomen and comparing the concentrations from each of the analyte sensor devices. Determining the concentration of an analyte using two or more analyte sensor devices positioned at different locations of the abdomen and comparing concentration values obtained by each analyte sensor device may be used to improve the accuracy or precision of the acquired analyte concentration values or may be used to further calibrate one or more of the analyte sensor devices. By “comparing” is meant the analyte concentration values obtained from each analyte sensor device may be related to each other mathematically (e.g., by an algorithm) or may simply be visually compared by the user. For example, in some instances, values obtained from one of the analyte sensor device may be used to calibrate one or more of the other analyte sensor device. In other instances, values obtained from one of the analyte sensor device may be used to mathematically (e.g., by an algorithm) correct the values obtained by one or more of the other analyte sensor device. Depending on the location of the analyte sensor device (e.g., having involuntary muscle movement or intentionally applied movement), methods for positioning and obtaining an analyte concentration from the two or more sensors may follow the appropriate protocols as described in greater detail above.

### Systems for Determining an Analyte Concentration

Aspects of the present disclosure also include analyte monitoring systems for practicing the subject methods (e.g., determining the analyte concentration). The particular configuration of a system and other units used in the analyte monitoring system may depend on the use for which the analyte monitoring system is intended and the conditions under which the analyte monitoring system will operate. One embodiment of the analyte monitoring system includes a sensor configured for implantation into the subject. For example, implantation of the sensor may be made for implantation in subcutaneous tissue for testing analyte levels in interstitial fluid.

This level may be correlated and/or converted to analyte levels in blood or other fluids. The site and depth of implantation may affect the particular shape, components, and configuration of the sensor. Examples of suitable sensors for use in the analyte monitoring systems of the invention are described in U.S. Pat. No. 6,175,752, the disclosure of which is incorporated herein by reference.

Additional embodiments of analyte monitoring systems suitable for practicing methods of the present disclosure are described in U.S. Patent Nos., U.S. Pat. No. 6,134,461, U.S. Pat. No. 6,579,690, U.S. Pat. No. 6,605,200, U.S. Pat. No. 6,605,201, U.S. Pat. No. 6,654,625, U.S. Pat. No. 6,746,582, U.S. Pat. No. 6,932,894, U.S. Pat. No. 7,090,756, U.S. Pat. No. 5,356,786; U.S. Pat. No. 6,560,471; U.S. Pat. No. 5,262,035; U.S. Pat. No. 6,881,551; U.S. Pat. No. 6,121,009; U.S. Pat. No. 7,167,818; U.S. Pat. No. 6,270,455; U.S. Pat. No. 6,161,095; U.S. Pat. No. 5,918,603; U.S. Pat. No. 6,144,837; U.S. Pat. No. 5,601,435; U.S. Pat. No. 5,822,715; U.S. Pat. No. 5,899,855; U.S. Pat. No. 6,071,391; U.S. Pat. No. 6,377,894; U.S. Pat. No. 6,600,997; U.S. Pat. No. 6,514,460; U.S. Pat. No. 5,628,890; U.S. Pat. No. 5,820,551; U.S. Pat. No. 6,736,957; U.S. Pat. No. 4,545,382; U.S. Pat. No. 4,711,245; U.S. Pat. No. 5,509,410; U.S. Pat. No. 6,540,891; U.S. Pat. No. 6,730,200; U.S. Pat. No. 6,764,581; U.S. Pat. No. 6,503,381; U.S. Pat. No. 6,676,816; U.S. Pat. No. 6,893,545; U.S. Pat. No. 6,514,718; U.S. Pat. No. 5,262,305; U.S. Pat. No. 5,593,852; U.S. Pat. No. 6,746,582; U.S. Pat. No. 6,284,478; U.S. Pat. No. 7,299,082; U.S. Pat. No. 7,811,231; U.S. Pat. No. 7,822,557; U.S. Pat. No. 8,106,780; Patent Application Publication No. 2010/0198034; U.S. Patent Application Publication No. 2010/0324392; U.S. Patent Application Publication No. 2010/0326842 U.S. Patent Application Publication No. 2007/0095661; U.S. Patent Application Publication No. 2008/0179187; U.S. Patent Application Publication No. 2008/0177164; U.S. Patent Application Publication No. 2011/0120865; U.S. Patent Application Publication No. 2011/0124994; U.S. Patent Application Publication No. 2011/0124993; U.S. Patent Application Publication No. 2010/0213057; U.S. Patent Application Publication No. 2011/0213225; U.S. Patent Application Publication No. 2011/0126188; U.S. Patent Application Publication No. 2011/0256024; U.S. Patent Application Publication No. 2011/0257495; U.S. Patent Application Publication No. 2012/0157801, U.S. Patent Application Ser. No. 13/407,617, and U.S. Patent Application Ser. No. 13/526,136, the disclosures of each of which are incorporated herein by reference in their entirety. Moreover, methods of the present disclosure may be practiced using battery-powered or self-powered analyte sensors, such as those disclosed in U.S. Patent Application Publication No. 2010/0213057, incorporated herein by reference in its entirety.

### Experimental

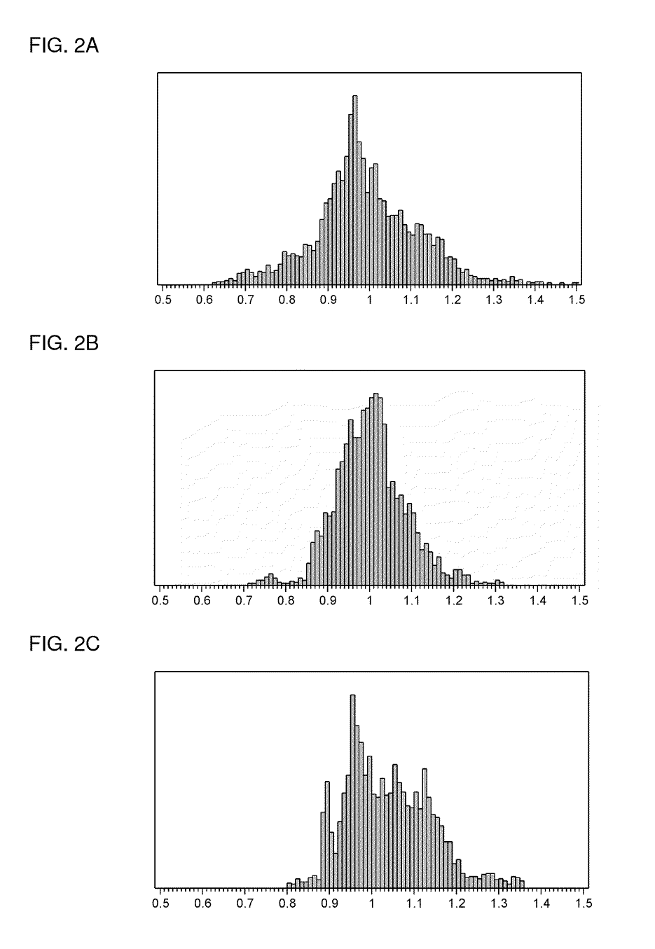

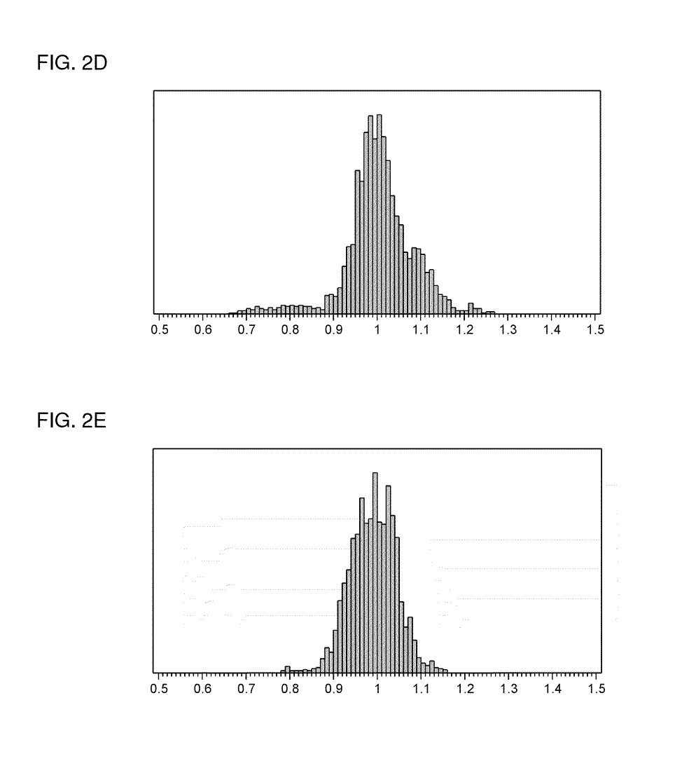

The histograms from paired and normalized commercially available continuous glucose monitors positioned at different locations on the abdomen are shown in FIGS. 2a-d. The current of each sensor was normalized by dividing its instantaneous value by its average value over the entire test. The ratio (normalized instantaneous current of sensor 1)/(normalized instantaneous current of sensor 2) was calculated for each minute, then the calculated current ratios were placed into 0.02 wide bins and their number in each bin was counted.

FIGS. 2a-e depict histograms of the ratio distribution of the data acquired from paired sensors where both sensors are positioned on the same person. FIGS. 2a: sensor 1 positioned near the apex (5.5 cm from the midline) of the abdomen and sensor 2 positioned on the calf just below the center of the inside of the knee. FIG. 2b: sensor 1 positioned to the far right (25.5 cm to the right of the midline) from the apex of the abdomen, and sensor 2 positioned to the near right (5.5 cm to the right of the midline) from the apex of the abdomen. FIG. 2c: sensor 1 positioned to the far left (25.5 cm to the left of the midline) from the apex of the abdomen and sensor 2 positioned to the near left (5.5 cm to the left of the midline) from the apex of the abdomen. FIG. 2d: sensor 1 positioned to the far right (25.5 cm to the right of the midline) from the apex of the abdomen, and sensor 2 positioned to the far left (25.5 cm to left of the midline) from the apex of the abdomen. FIG. 2e: sensor 1 positioned to the near left (5.5 cm to the left of the midline) from the apex of the abdomen, and sensor 2 positioned to the near right (5.5 cm to the right of the midline) from the apex of the abdomen.

The deviation from a normal or Gaussian distribution is a measure of the temporal dissimilarity of the interstitial fluid found at each particular location. As such, dissimilarity between the interstitial fluid found in each location would result in different determined analyte concentrations depending on the location of the positioned analyte sensor device. As depicted in FIGS. 2d and 2e, the distribution is close to normal, or Gaussian, for paired sensors where a first sensor is positioned to the far left from the apex of the abdomen and a second sensor positioned to the far right from the apex of the abdomen (e.g., FIG. 2d) and where a first sensor is positioned to the near left from the apex of the abdomen and a second sensor positioned to the near right from the apex of the abdomen (e.g., FIG. 2e). However, the distribution is much broader and is not Gaussian for paired sensors where a first sensor is positioned at the apex of the abdomen and a second sensor is positioned on the inside of the knee (e.g., FIG. 2a). This demonstrates that there is a temporal dissimilarity between the interstitial fluid on the inside of the knee with the interstitial fluid at the abdomen Likewise, there is a slightly broader and less normal distribution for paired sensors on the abdomen such as where a first sensor is positioned to the far left from the apex of the abdomen and a second sensor is positioned to the near left from the apex of the abdomen (e.g. FIG. 2c). Similarly, the distribution is much broader and is a less normal distribution for paired sensors where a first sensor is positioned to the far right from the apex of the abdomen and a second sensor is positioned to the near right from the apex of the abdomen (e.g., FIG. 2b). This demonstrates that there is a slight dissimilarity between the interstitial fluid far from the abdominal apex and near the abdominal apex.

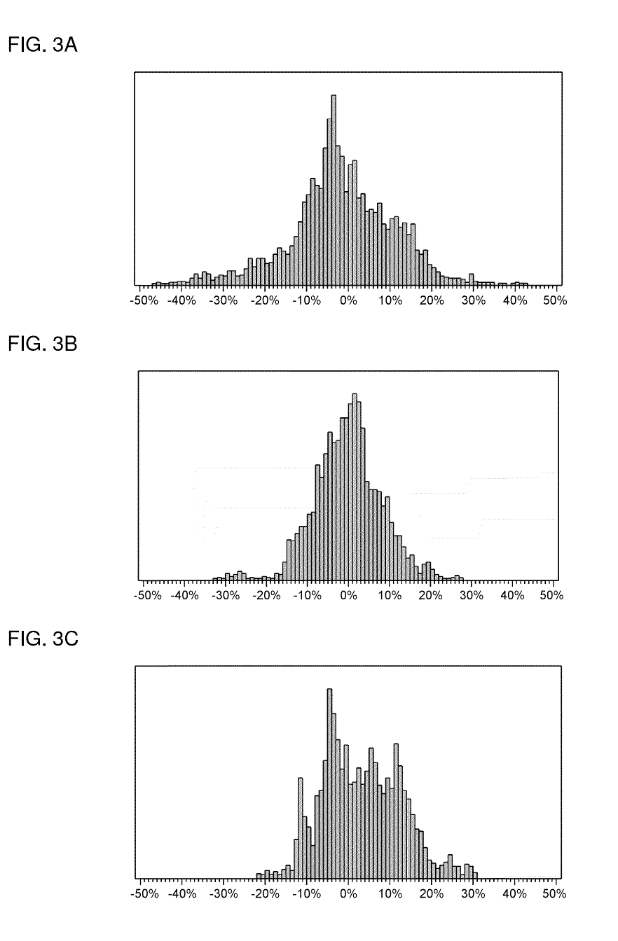

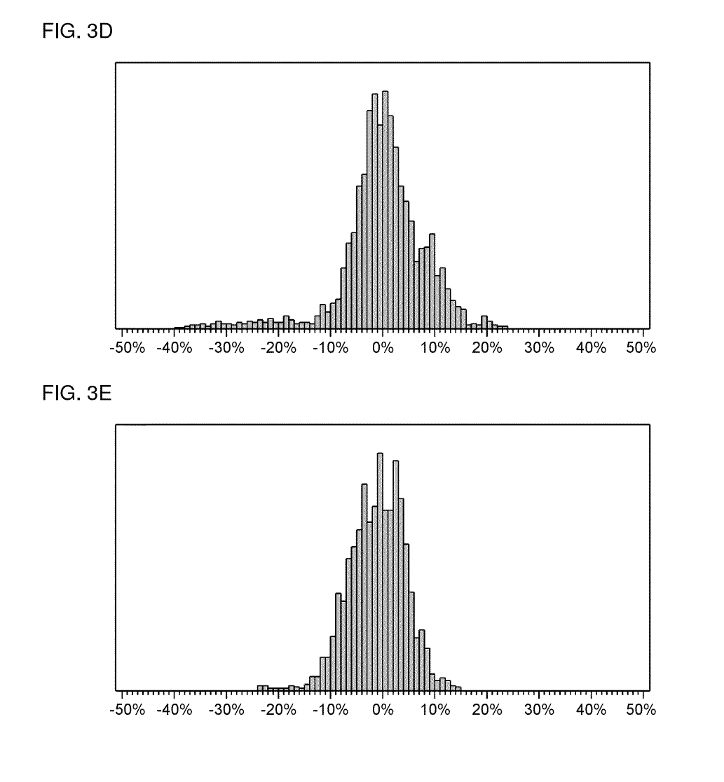

FIGS. 3a-d depict the normalized paired sensor output differences defined by equation (1): (Sens₁−Sens₂)/½(Sens₁+Sens₂) for the sensors positioned as discussed above for FIGS. 2a-e. For two error-free sensors in two temporally similarly behaving ISFs the distribution would be an infinitesimally narrow line, vertical to the x-axis, at x=0. For two sensors with a finite measurement error, residing in temporally similarly behaving ISFs, the width of the distribution is expected to increase and the height of the distribution is expected to decrease as the measurement error increases; the distribution would remain normal i.e. Gaussian, even for large measurement errors. For two sensors residing in two temporally dissimilar fluids the distribution is not expected to be Gaussian, irrespective of the measurement error. The deviation from Gaussian distribution is a measure of the temporal dissimilarity of the two ISFs.

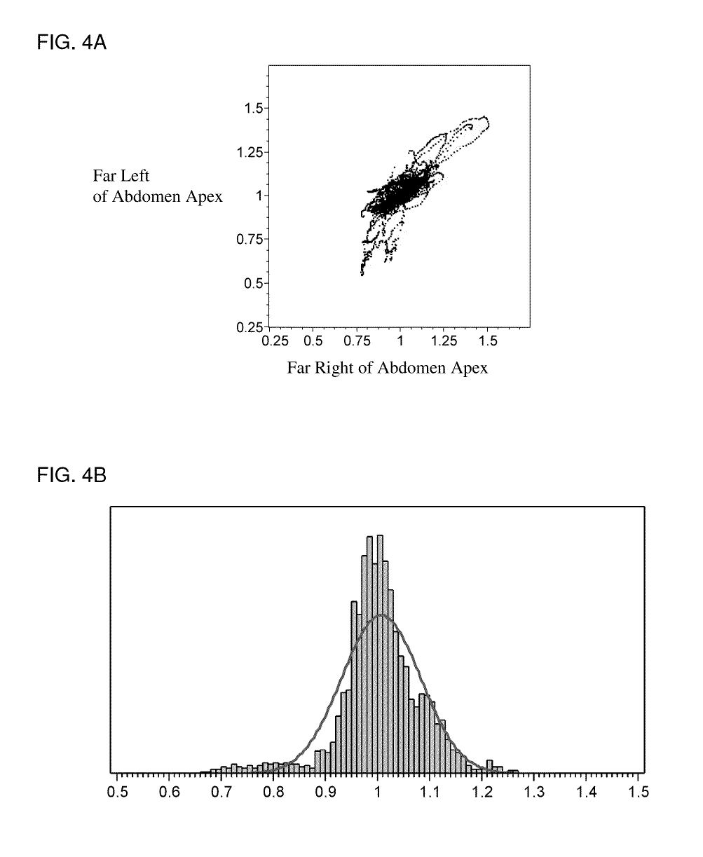

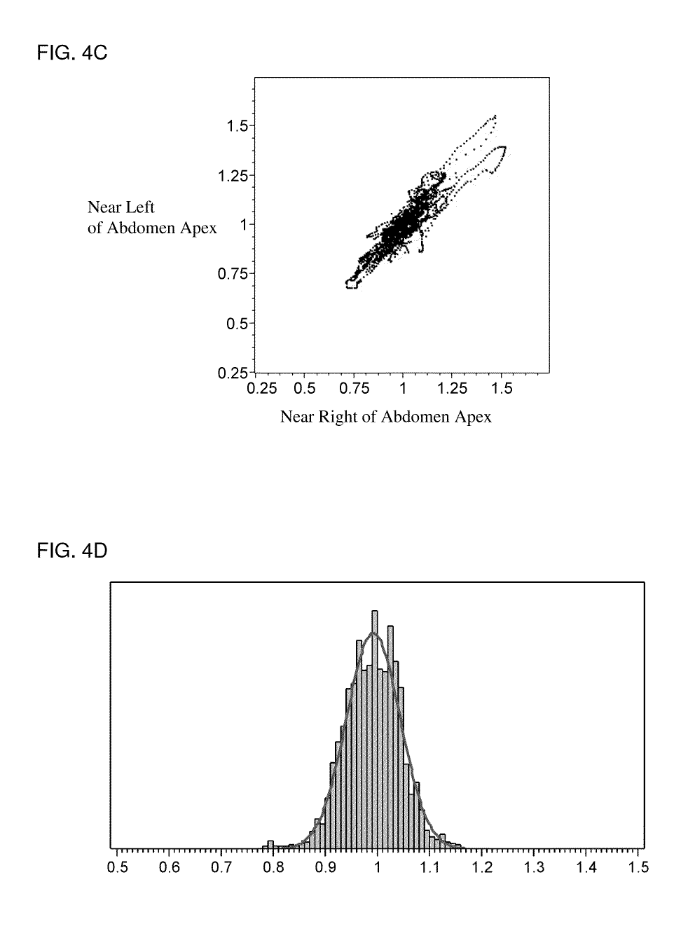

As illustrated in FIGS. 3a-e, the distributions for two sensors for the two sensors positioned equidistant to the right and left of the abdominal apex (e.g., both near or far from the abdominal apex). They are, however, much broader and are not Gaussian for two sensors with one residing in the abdomen and the other in the back of the knee. Likewise, the distributions are broader and are not Gaussian for two sensors where one is positioned far from the abdominal apex and the other is positioned near the abdominal apex. Consequently, the glycemia of the ISF of those sites where CGM sensors are now worn, namely the upper arm side facing away from the chest, are inferior to those positioned on the abdomen, meaning that the blood glycemia estimated when the outer upper arm is the site of the implanted sensor is inferior to the estimate of the blood glycemia based on readings with a sensor located on the abdomen (e.g., near the abdominal apex). Table 1 below, summarizes the temporal similarity of interstitial fluid found at a particular location as illustrated in FIGS. 3 and 4.

TABLE 1

| Sensors Position | | | | | |
|---|---|---|---|---|---|
| Location | FIG. | Mean | Std. Dev. | Skew | Kurtosis |
| Sensor 1: Apex of | 2a | 0.991 | 0.121 | 0.233 | 0.823 |
| abdomen | | | | | |
| Sensor 2: Behind | | | | | |
| the Knee | | | | | |
| Sensor 1: Far right | 2b | 1.00 | 0.0817 | 0.232 | 0.910 |
| abdomen (25.5 cm) | | | | | |
| Sensor 2: Near right | | | | | |
| abdomen (5.5 cm) | | | | | |
| Sensor 1: Far left | 2c | 1.04 | 0.0996 | 0.522 | 0.0154 |
| abdomen (25.5 cm) | | | | | |
| Sensor 2: Near left | | | | | |
| abdomen (5.5 cm) | | | | | |
| Sensor 1: Far right | 2d | 1.01 | 0.0755 | −0.570 | 2.66 |
| abdomen (25.5 cm) | | | | | |
| Sensor 2: Far left | | | | | |
| abdomen (25.5 cm) | | | | | |
| Sensor 1: Near right | 2e | 0.991 | 0.0518 | −0.149 | 0.385 |
| abdomen (5.5 cm) | | | | | |
| Sensor 2: Near left | | | | | |
| abdomen (5.5 cm) | | | | | |
| Sensor 1: Apex of | 3a | −1.64% | 12.3% | −0.260 | 0.754 |
| abdomen | | | | | |
| Sensor 2: Behind | | | | | |
| the Knee | | | | | |
| Sensor 1: Far right | 3b | −0.104% | 8.15% | −0.123 | 1.02 |
| abdomen (25.5 cm) | | | | | |
| Sensor 2: Near right | | | | | |
| abdomen (5.5 cm) | | | | | |
| Sensor 1: Far left | 3c | 3.18% | 9.44% | 0.280 | −0.316 |
| abdomen (25.5 cm) | | | | | |
| Sensor 2: Near left | | | | | |
| abdomen (5.5 cm) | | | | | |
| Sensor 1: Far right | 3d | 0.290% | 7.76% | −1.10 | 4.08 |
| abdomen (25.5 cm) | | | | | |
| Sensor 2: Far left | | | | | |
| abdomen (25.5 cm) | | | | | |
| Sensor 1: Near right | 3e | −1.05% | 5.26% | −0.340 | 0.659 |
| abdomen (5.5 cm) | | | | | |
| Sensor 2: Near left | | | | | |
| abdomen (5.5 cm) | | | | | |

FIGS. 4a-b depict correlation of normalized signals and the distribution of the ratio of normalized signals for symmetrically positioned sensors on the far left (25.5 cm to the left of the midline) abdomen and the far right (25.5 cm to the right of the midline) abdomen. In this embodiment, sensors were positioned at the level of navel, equidistant from the midline of the body. Localized involuntary muscle movement in the form of normal respiration (i.e., breathing) provides movement of interstitial fluid at the location of these sensors. FIGS. 4c-d depict correlation of normalized signals and the distribution of the ration of normalized signals for symmetrically positioned sensors on the near left (5.5 cm to the left of the midline) abdomen and the near right (5.5 cm to the right of the midline) abdomen. In this embodiment, sensors were positioned at the level of navel, laterally equidistant from the midline of the body. Localized involuntary muscle movement in the form of normal respiration (i.e., breathing) provides movement of interstitial fluid at the location of these sensors. As depicted by FIGS. 4a-d, involuntary muscle movement due to normal breathing produced similar movement of ISF such that the outputs of symmetrically placed left and right sensors show similar data values.

The data illustrated in FIG. 5 is summarized in Table 2 below:

TABLE 2

| Sensors Position | | | | R- |
|---|---|---|---|---|
| Location | FIG. | Slope | Intercept | Squared |
| Sensor 1: Far right | 4a | 0.859 | 0.146 | 0.628 |
| abdomen (25.5 cm) | | | | |
| Sensor 2: Far left | | | | |
| abdomen (25.5 cm) | | | | |
| Sensor 1: Near right | 4c | 0.948 | 0.0428 | 0.823 |
| abdomen (5.5 cm) | | | | |
| Sensor 2: Near left | | | | |
| abdomen (5.5 cm) | | | | |

Table 3 summarizes data obtained by continuous glucose monitoring positioned on a part of the body which does not experience involuntary muscle movement—the calf. As illustrated, in the absence of movement (e.g., during sleep), there was a weaker correlation between glucose measurements in the interstitial fluid.

TABLE 3
Asleep - No Movement (e.g., in supine position)

| R² | Slope | Intercept |
|---|---|---|
| 0.41 | 0.4 | 0.51 |
| 0.35 | 0.78 | 0.22 |

Table 4 summarizes data obtained by continuous glucose monitoring positioned at locations on the body that experience localized involuntary movement sufficient to provide for movement of interstitial fluid at the location on the body while the subject is awake. As illustrated, while the subject is awake, normal breathing resulted in a strong correlation between glucose measurements obtained in interstitial fluid from the CGM sensors and the glucose measurements as obtained by blood.

TABLE 4

| Positioning Location | R² | Slope | Intercept |
|---|---|---|---|
| Just Below Diaphragm (2.5 cm) | 0.89 | 1.00 | 0.01 |
| Just Below Diaphragm (1.0 cm) | 0.84 | 0.83 | 0.19 |
| Below Diaphragm (6.5 cm) | 0.84 | 0.9 | 0.09 |
| Aligned with Navel (12.5 cm below | 0.8 | 0.87 | 0.15 |
| diaphragm) | | | |
| Avg. Respiration - Moved Abdomen | 0.85 | 0.91 | 0.09 |
| Center, Awake | | | |

Table 5 summarizes data obtained by continuous glucose monitoring positioned at locations on the body that experience localized involuntary movement sufficient to provide for movement of interstitial fluid at the location on the body while the subject is asleep. As illustrated, while the subject is asleep, normal breathing resulted in a strong correlation between glucose measurements obtained in interstitial fluid from the CGM sensors and the glucose measurements as obtained by blood.

TABLE 5

| Positioning Location | R² | Slope | Intercept |
|---|---|---|---|
| Just Below Diaphragm (2.5 cm) | 0.67 | 0.75 | 0.25 |
| Just Below Diaphragm (1.0 cm) | 0.56 | 1.08 | −0.08 |
| Below Diaphragm (6.5 cm) | 0.89 | 1.14 | −0.13 |
| Aligned with Navel (12.5 cm below | 0.85 | 1.01 | −0.01 |
| diaphragm) | | | |
| Avg. Respiration - Moved Abdomen | 0.74 | 1.01 | −0.01 |
| Center, Asleep | | | |

Table 6 summarizes data obtained by continuous glucose monitoring positioned at locations on the body that experience localized involuntary movement sufficient to provide for circulation of interstitial fluid at the location on the body while the subject is asleep and awake. As illustrated, while the subject is asleep and awake, normal breathing resulted in a strong correlation between glucose measurements obtained in interstitial fluid from the CGM sensors and the glucose measurements as obtained by blood.

TABLE 6

| Positioning Location | R² | Slope | Intercept |
|---|---|---|---|
| Just Below Diaphragm (2.5 cm) | 0.81 | 0.91 | 0.09 |
| Just Below Diaphragm (1.0 cm) | 0.72 | 0.89 | 0.11 |
| Below Diaphragm (6.5 cm) | 0.86 | 0.98 | 0.02 |
| Aligned with Navel (12.5 cm below | 0.82 | 0.87 | 0.14 |
| diaphragm) | | | |
| Avg. Respiration - Moved Abdomen | 0.80 | 0.93 | 0.07 |
| Center, Awake and Asleep | | | |

FIGS. 5a-b illustrate the calculation and correlation between the scaled current from a continuous glucose monitor for glucose measurements obtained in interstitial fluid with glucose values as obtained by blood using commercially available in vitro blood glucose test strips. FIG. 5a depicts the correlation between scaled currents obtained by the CGM sensor device positioned near the apex of the abdomen with blood glucose concentration. Blood glucose may be calculated from the scaled current according to Equation (1):

Blood Glucose (BG)=Scaled Current (SC)×(Average BG)/(Average SC).

FIG. 5b depicts the correlation between blood glucose as determined using Equation (1) from the scaled current (from continuous glucose monitor sensors positioned at different locations on the body which experience localized involuntary muscle movement) and blood glucose as determined using a blood glucose meter.

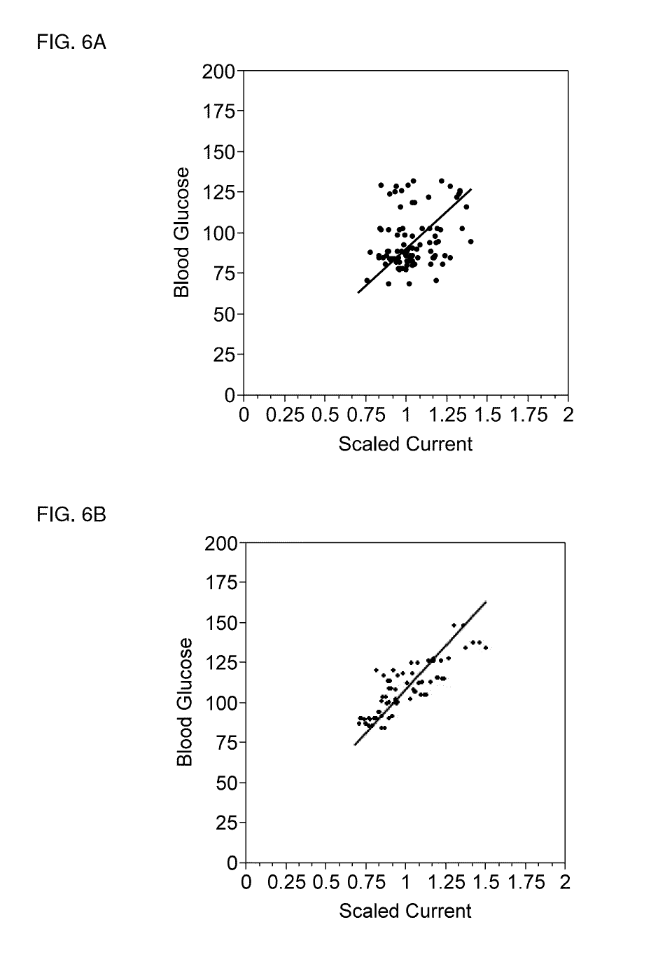

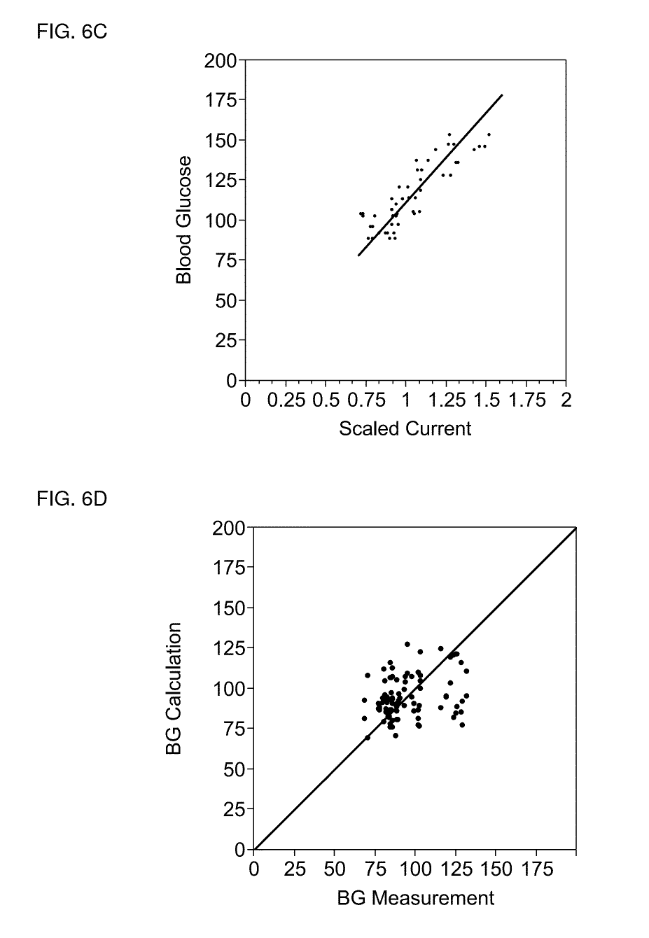

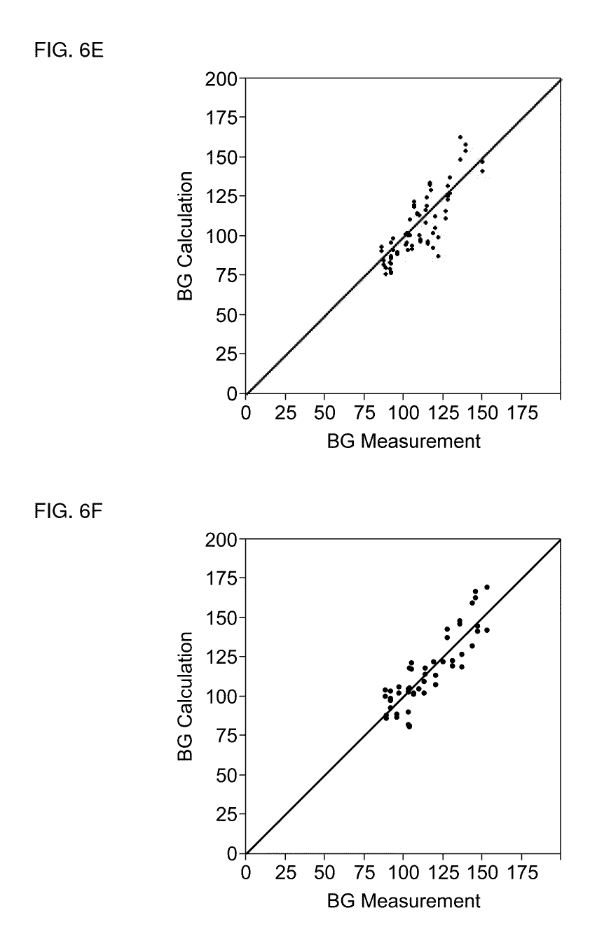

FIGS. 6a-c depict the correlation between the scaled current (from continuous glucose monitor sensors positioned at different locations on the abdomen which experience localized involuntary muscle movement) and blood glucose as determined using a blood glucose meter. FIGS. 6d-f depict the correlation between blood glucose as determined using Equation (1) from the scaled current from continuous glucose monitor sensors positioned at different locations on the body which experience localized involuntary muscle movement and blood glucose as determined using a blood glucose meter. Continuous glucose monitor sensors are positioned in FIGS. 6a and 6d: far from the abdominal apex (25.5 cm from the midline), in FIGS. 6b and 6e: near the abdominal apex (e.g., 5.5 cm from the midline); in FIGS. 6c and 6f: at the abdominal apex. As illustrated by the data in FIGS. 6a-f, there is a strong correlation between glucose values determined from scaled currents by continuous glucose monitoring sensors in the interstitial fluid and glucose values determined by a glucose meter from blood in the sensors which are positioned near the abdominal apex (i.e., 5.5 cm from the midline). There is a weaker correlation between glucose values determined from scaled currents by continuous glucose monitoring sensors in the interstitial fluid and glucose values determined by a glucose meter from blood in the sensors which are positioned far from the abdominal apex (i.e., 25.5 cm from the midline). As such, FIG. 6 demonstrates that locations which experience greater localized involuntary movement provide more accurate glucose measurements.

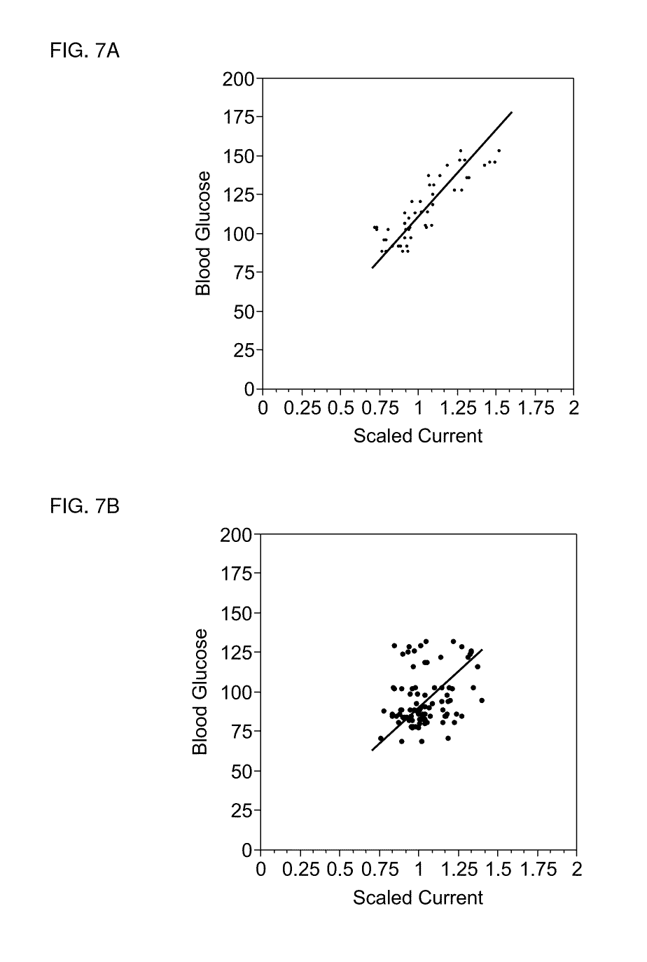

FIGS. 7a-c depict the correlation between blood glucose as determined using Equation (1) from scaled current and blood glucose as determine using a blood glucose meter. Continuous glucose monitor sensors are positioned in FIG. 7a: at the abdominal apex; in FIG. 7b: near the abdominal apex (5.5 cm from the midline); and in FIG. 7c: far from the abdominal apex (25.5 cm from the midline). FIGS. 7a-b demonstrate that locations which experience greater localized involuntary movement provide more accurate glucose measurements.

As described in detail above, methods of the present disclosure help to reduce hypoglycemic alarms even during sleep or periods of little to no deliberate or voluntary muscle movement. Below is a comparison of non-limiting examples which illustrate that by employing methods and systems of the present disclosure, a much lower error between the interstitial fluid glycemia as compared to the blood glycemia is obtained, this lower error thus helping to prevent missed hypo- or hyper-glycemic alarms.

As described above, as much as 15% of interstitial fluid introduced into the interstitial space from arterioles is not rapidly cleared by the venules and is subsequently slowly cleared by the lymphatic system (e.g., 24 hours in the absence of movement). In some instances, blood glycemia can change at a rate of 2 mg/dL min⁻¹.

CASE 1: No involuntary movement at the location of the positioned sensor. 15% of the interstitial fluid remains stagnant. Blood glycemia decreases from 200 mg/dL at time=0 to 50 mg/dL at time=120 minutes. The measured interstitial fluid glycemia at time=120 minutes is (0.85)×(50 mg/dL)+(0.15)×(200 mg/dL)=72.5 mg/dL. Therefore, in the absence of involuntary movement to mix the interstitial fluid, the percentage error between the interstitial fluid glycemia as compared to the blood glycemia is 45%.

CASE 2: Involuntary movement present at the location of the positioned sensor according to methods of the present disclosure where the presence of involuntary movement enables clearing of the stagnant interstitial fluid. As such, involuntary movement replaces in 2 minutes the interstitial fluid which has not been cleared by the venules. The error between the interstitial fluid glycemia as compared to the blood glycemia in which involuntary movement replaces the interstitial fluid in 2 minutes is (2 minutes)×(150 mg/dL decrease)/120 minutes=2.5 mg/dL. Therefore the percentage error between the interstitial fluid glycemia as compared to the blood glycemia where there is involuntary movement is only 5%.

CASE 3: No involuntary movement at the location of the positioned sensor. 15% of the interstitial fluid remains stagnant. Blood glycemia increases from 70 mg/dL to 250 mg/dL in 180 minutes. The measured interstitial fluid glycemia at time=180 minutes is (0.85)×(250 mg/dL)+(0.15)×(70 mg/dL)=223 mg/dL. Therefore, in the absence of involuntary movement to mix the interstitial fluid, the percentage error between the interstitial fluid glycemia as compared to the blood glycemia is 10%.

CASE 4: Involuntary movement present at the location of the positioned sensor according to methods of the present disclosure where the presence of involuntary movement enables clearing of the stagnant interstitial fluid. As such, involuntary movement replaces in 2 minutes the interstitial fluid which has not been cleared by the venules. The error between the interstitial fluid glycemia as compared to the blood glycemia in which involuntary movement replaces the interstitial fluid in 2 minutes is (2 minutes)×(180 mg/dL decrease)/180 minutes=2 mg/dL. Therefore the percentage error between the interstitial fluid glycemia as compared to the blood glycemia where there is involuntary movement is only about 1%.

By positioning an analyte sensor device at a location on the abdomen which experiences involuntary movement, a much lower error between the interstitial fluid glycemia as compared to the blood glycemia is obtained which helps to prevents missed hypo- or hyper-glycemic alarms.

The present description should not be considered limited to the particular examples described above, but rather should be understood to cover all aspects as fairly set out in the attached claims. Various modifications, equivalent processes, as well as numerous structures to which the transition metal complexes may be applicable will be readily apparent to those of skill in the art upon review of the instant specification.

That which is claimed is:

1. A method of determining a concentration of an analyte, the method comprising:
determining a location on a subject's abdomen that experiences localized involuntary muscle movement sufficient to provide for mixing of circulating and non-circulating interstitial fluid in a subcutaneous space of the location;
positioning an analyte sensor device subcutaneously at the determined location on the subject's abdomen; and
determining an analyte concentration in the interstitial fluid using the positioned analyte sensor.

2. The method according to claim 1, wherein the determining of the location on the subject's abdomen comprises:
locating the navel of the subject;
locating the lowest point of the ribcage of the subject; and
identifying along the midline of the abdomen of the subject a location that is equidistant from the navel and the lowest point of the ribcage.

3. The method according to claim 2, wherein positioning comprises positioning the analyte sensor 12 cm or less to the left or to the right from the midline of the abdomen of the subject.

4. The method according to claim 3, wherein positioning comprises positioning the analyte sensor 6 cm or less to the left or to the right from the midline of the abdomen of the subject.

5. The method according to claim 1, wherein the location experiences the involuntary muscle movement as a result of respiration.

6. The method according to claim 1, wherein the location is below the diaphragm.

7. The method according to claim 1, wherein the location is 12 cm or less to the left or 12 cm or less to the right of the apex of the abdomen.

8. The method according to claim 1, wherein the location is at the apex of the abdomen.

9. The method according to claim 1, wherein the location is above the navel.

10. The method according to claim 1, wherein at least 95% of the analyte concentration values determined in the interstitial fluid is within 5% of an analyte concentration determined in blood.

11. The method according to claim 1, wherein at least 99% of the analyte concentration values determined in the interstitial fluid is within 5% of an analyte concentration determined in blood.

12. The method according to claim 1, wherein the localized involuntary muscle movement at the location has a total displacement rate from about 100 to 500 mm per minute.

13. The method according to claim 1, wherein the localized involuntary muscle movement at the location is due to the subject taking from about 10 to 20 breaths per minute.

14. The method according to claim 1, wherein the method further comprises determining the analyte concentration while the subject is asleep.

15. The method according to claim 1, wherein the method further comprises determining the analyte concentration while the subject is awake.

16. The method according to claim 1, further comprising monitoring the localized involuntary muscle movement sufficient to provide for mixing of circulating and non-circulating interstitial fluid in a subcutaneous space of the location, during the sensor wear period.

17. The method according to claim 1, wherein the positioning an analyte sensor comprises:
placing a housing adapted for placement on the surface of skin having a bottom surface for contacting with the skin and wherein the housing comprises;
an electrochemical sensor having a portion within the housing and a portion exterior to the housing and having a length to permit insertion of the second portion beneath the surface of the skin; and
an adhesive disposed on the bottom surface of the housing to attach the housing to the surface of the skin.

18. The method according to claim 1, wherein positioning comprises:
contacting an insertion device coupled with the analyte sensor device to the skin of the subject;
inserting at least a portion of the electrochemical sensor subcutaneously beneath the surface of the skin at the location on the body of the subject using the insertion device; and
decoupling the insertion device from the analyte sensor unit.

19. The method according to claim 18, wherein the electrochemical sensor is inserted to a depth of about 2.0 to about 8.0 mm beneath the surface of the skin.

20. The method according to claim 17, wherein the electrochemical sensor comprises:
a working electrode comprising an analyte responsive enzyme and a mediator; and
a counter electrode.

21. The method according to claim 20, wherein the analyte is glucose and wherein the analyte responsive enzyme is glucose oxidase or glucose dehydrogenase.

22. The method according to claim 1, wherein the method further comprises displaying the analyte concentration.

23. A method of determining a concentration of an analyte during sleep, the method comprising:
determining a location on a subject's abdomen that experiences localized involuntary muscle movement during sleep sufficient to provide for mixing of circulating and non-circulating interstitial fluid in a subcutaneous space of the location
positioning an analyte sensor device subcutaneously at the determined location on the subject's abdomen; and
determining an analyte concentration in the interstitial fluid using the positioned sensor.

24. The method according to claim 23, wherein determining the location on the subject's abdomen comprises:
locating the navel of the subject;
locating the lowest point of the ribcage of the subject;
identifying along the midline of the abdomen of the subject a location that is equidistant from the navel and the lowest point of the ribcage.

25. The method according to claim 24, wherein positioning comprises positioning the analyte sensor 12 cm or less to the left or to the right from the midline of the abdomen of the subject.

26. The method according to claim 25, wherein positioning comprises positioning the analyte sensor 6 cm or less to the left or to the right from the midline of the abdomen of the subject.

27. The method according to claim 23, wherein the location experiences the involuntary muscle movement as a result of respiration.

28. The method according to claim 23, wherein the location is below the diaphragm.

29. The method according to claim 23, wherein the location is 12 cm or less to the left or 12 cm or less to the right of the apex of the abdomen.

30. The method according to claim 23, wherein the location is at the apex of the abdomen.

31. The method according to claim 23, wherein the location is above the navel.

32. The method according to claim 23, wherein at least 95% of the analyte concentration values determined in the interstitial fluid is within 5% of an analyte concentration determined in blood.

33. The method according to claim 23, wherein at least 99% of the analyte concentration values determined in the interstitial fluid is within 5% of an analyte concentration determined in blood.

34. The method according to claim 23, wherein the localized involuntary muscle movement at the location has a total displacement rate from about 100 to 500 mm per minute.

35. The method according to claim 23, wherein the localized involuntary muscle movement at the location is due to the subject taking from about 10 to 20 breaths per minute.

36. The method according to claim 23, wherein the analyte sensor device comprises:
a housing adapted for placement on the surface of skin having a bottom surface for contacting with the skin and wherein the housing comprises:
an electrochemical sensor having a portion within the housing and a portion exterior to the housing and having a length to permit insertion of the second portion beneath the surface of the skin; and
an adhesive disposed on the bottom surface of the housing to attach the housing to the surface of the skin.

37. The method according to claim 36, wherein positioning comprises:
contacting an insertion device coupled with the analyte sensor device to the skin of the subject;
inserting at least a portion of the electrochemical sensor subcutaneously beneath the surface of the skin at the location on the body of the subject using the insertion device; and
decoupling the insertion device from the analyte sensor unit.

38. The method according to claim 37, wherein the electrochemical sensor is inserted to a depth of about 2.0 to about 8.0 mm beneath the surface of the skin.

39. The method according to claim 36, wherein the electrochemical sensor comprises:
a working electrode comprising an analyte responsive enzyme and a mediator; and
a counter electrode.

40. The method according to claim 39, wherein the analyte is glucose and wherein the analyte responsive enzyme is glucose oxidase or glucose dehydrogenase.

41. The method according to claim 23, wherein the method further comprises displaying the analyte concentration.
